(19) United States
(12) Patent Application Publication
(10) Pub. No.: US 2008/0020075 A1
(43) Pub. Date: Jan. 24, 2008

(54) Topical Cypress Essential Oil Composition for Stretch Marks

(76) Inventor: Lelee Mehregani, Western Australia (AU)

Correspondence Address: BACHMAN & LAPOINTE, P.C., 900 CHAPEL STREET, SUITE 1201, NEW HAVEN, CT, 06510, US

(21) Appl. No.: 11/579,973

(22) PCT Filed: May 9, 2005
(86) PCT No.: PCT/AU05/00655

§ 371 Date: Nov. 8, 2006

(30) Foreign Application Priority Data
May 11, 2004 (AU) 2004902458

(51) Int. Cl. A61K 36/14; A61P 17/16

(52) U.S. Cl. 424/770

(57) ABSTRACT

A method for reducing or minimizing the appearance of striae distensae lesions by topically applying a composition comprising sweet almond oil, wheat germ oil, olive oil, castor oil, and cypress essential oil.

### CROSS REFERENCE TO RELATED APPLICATIONS

This case is the U.S. national phase of International Application No. PCT/AU2005/000655 filed May 9, 2005 which claims priority of Australian Application No. 2004902458 filed May 11, 2004.

### FIELD OF THE INVENTION

The present invention relates to a topically applied composition, in particular to a topically applied composition for treatment of striae distensae lesions, also knowns as stretch marks.

### BACKGROUND OF THE INVENTION

Striae distensae lesions, also known as stretch marks, typically occur on the abdomen, breasts, buttocks, hips, and thighs. They are thought to arise due to weakening of the elastic skin tissues during pregnancy, rapid growth spurts in adolescence, and periods of excessive weight change. Their appearance can also be associated with topical or prolonged treatment with corticosteroids.

It is estimated that approximately fifty percent of pregnant women will develop striae distensae lesions throughout the course of their pregnancy.

Although striae distensae lesions are not regarded as a medical problem, their cosmetic appearance can cause considerable distress, particularly if the affected area is widespread. Furthermore, to date there has been no known treatment that minimizes or reduces their appearance although the appearance of the lesions does become less pronounced over prolonged periods of time.

The present invention seeks to overcome or at least alleviate striae distensae lesions and/or their appearance.

It is to be understood that, although prior art use and publications may be referred to herein, such reference does not constitute an admission that any of these form a part of the common general knowledge in the art, in Australia or any other country. Summary of the Invention

In one aspect, the invention features a topically applied composition for treatment of striae distensae lesions, the composition comprising a pharmaceutically or cosmetically acceptable vehicle for topical application and an effective amount of cypress essential oil.

Typically, the cypress essential oil is present in an amount up to 5% by volume, preferably between 1.0-4.0% by volume.

In one embodiment of the invention the pharmaceutically or cosmetically acceptable vehicle comprises a carrier oil.

Examples of carrier oils include, but are not limited to, sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, and vegetable oils such as for example safflower oil, sunflower oil, canola oil, soybean oil, and peanut oil, and combinations thereof.

The preferred carrier oil is a mixture of sweet almond oil, wheat germ oil, olive oil, and castor oil.

In the preferred embodiment the topically applied composition comprises sweet almond oil, wheat germ oil, olive oil, castor oil, and cypress essential oil. Preferably the topically applied composition comprises sweet almond oil in the range of 20-60% v/v, wheat germ oil in the range of 20-55% v/v, olive oil in the range of 15- 45% v/v, castor oil in the range of 2-10%v/v, and cypress essential oil in the range of up to 5% v/v, with the balance of the composition at 100%.

In alternative embodiments of the invention the pharmaceutically or cosmetically acceptable vehicles comprise lotions, creams, ointments, gels, and solid formulations such as for example a wax-based stick.

In one embodiment, the composition further comprises other pharmaceutically active agents in addition to cypress essential oil, provided that they do not detrimentally affect the desired effect on the treated area afforded by the cypress essential oil.

In another aspect, the invention features a method of treating striae distensae lesions to improve the appearance thereof comprising topically applying to an area affected by the lesions a composition comprising a pharmaceutically or cosmetically acceptable vehicle for topical treatment and an effective amount of cypress essential oil.

In the preferred embodiment, the composition is topically applied to an area affected by striae distensae lesions at least once a week, preferably one or twice a day. Typically, the composition is topically applied to the affected area until the appearance of the striae distensae lesions has been improved, reduced, or minimized to the satisfaction of the user.

In a further aspect, the invention features a method of improving the appearance of the striae distensae lesions comprising topically applying to an area affected by the lesions a composition comprising a pharmaceutically or cosmetically acceptable vehicle for topical treatment and an effective amount of cypress essential oil.

In an alternative aspect, the invention features a topically applied composition when used to prepare a dermopharmaceutical or cosmetic to improve the appearance of the striae distensae lesions, the topically applied composition comprising a pharmaceutically or cosmetically acceptable vehicle for topical treatment and an effective amount of cypress essential oil.

The topically applied compositions of the present invention are prepared by mixing the cypress essential oil and, optionally, other pharmaceutically active agents with the pharmaceutically or cosmetically acceptable vehicle.

In the description and the claims of the invention, except where the context requires otherwise due to express language or necessary implication, the words “comprise” or variations such as “comprises” or “comprising” are used in an inclusive sense, i.e. to specify the presence of the stated features, but not to preclude the presence or addition of further features in various embodiments of the invention.

Unless otherwise specifically noted, all percentages used in the following specification and claims are by volume.

### DETAILED DESCRIPTION OF THE PREFERRED EMBODIMENTS OF THE INVENTION

Before the preferred embodiment of the present apparatus is described, it is understood that this invention is not limited to the particular materials described, as these may vary. It is also to be understood that the terminology used herein is for the purpose of describing the particular embodiment only, and is not intended to limit the scope of the present invention in any way. It must be noted that as used herein, the singular forms “a”, “an”, and “the” include plural reference unless the context clearly dictates otherwise. Unless defined otherwise, all technical and scientific terms used herein have the same meanings as commonly understood by one of ordinary skill in the art to which this invention belongs.

A topically applied composition for treatment of striae distensae lesions to improve the appearance thereof is provided.

The terms “topically applied”, “topically applying”, or “topical application” as used herein refer to directly laying on or spreading on outer skin using an applicator, such as a wipe or a cotton swab, or by hand.

In referring to the phrases “improving the appearance”, “to improve the appearance”, or “an (marked) improvement in the appearance” throughout this description, it is inferred that the colour of the lesions changes from an inflamed reddish appearance to pink, through to a pale skin tone colour, and/or the width and/or the depth of the lesions narrows such that the appearance of the lesions in the affected area become less pronounced than before the treatment period, during which treatment period the composition of the present invention is regularly applied to an affected area, commenced.

The topically applied composition comprises a pharmaceutically or cosmetically acceptable vehicle for topical treatment and an effective amount of cypress essential oil.

Cypress essential oil is derived from Cupressus semperivirens, typically by steam distillation of the needles thereof. Typically, the cypress essential oil is present in the composition in an amount up to 5% by volume, preferably between 1.0-4.0% by volume. The upper limit envisaged for the maximum amount of cypress essential oil in the topically applied composition is limited by the appearance of toxic effects associated with cypress essential oil, which are thought to manifest at concentrations of cypress essential oil of >10% by volume.

The term “pharmaceutically or cosmetically acceptable” as used herein refers to pharmaceutically active agents or inert ingredients which are suitable for use in contact with the skin without undue toxicity, incompatibility, instability, irritation, allergic response, and the like, commensurate with a reasonable benefit/risk ratio.

The term “effective amount” as used herein refers to an amount of composition sufficient to significantly induce an improvement in the appearance of the striae distensae lesions of the affected area that is treated, but low enough to avoid serious side effects. The effective amount of the composition will vary with the age and physical condition of the patient in need of treatment, the extent of the area that requires treatment, the severity of the striae distensae lesions, the duration of the treatment, the particular pharmaceutically or cosmetically acceptable vehicle used, and like factors.

Typically, the pharmaceutically or cosmetically acceptable vehicle comprises at least 95% volume of the topically applied composition of the present invention.

In one embodiment of the invention that pharmaceutically or cosmetically acceptable vehicle comprises a carrier oil. Examples of carrier oils include, but are not limited to, sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, and vegetable oils such as for example safflower oil, sunflower oil, canola oil, soybean oil, and peanut oil, and combinations thereof.

In the preferred embodiment of the invention, the preferred carrier oil is a mixture of sweet almond oil, wheat germ oil, olive oil, castor oil, such that the topically applied composition comprises sweet almond oil, wheat germ oil, olive oil, castor oil, and cypress essential oil.

Preferably the topically applied composition comprises sweet almond oil in the range of 20-60% v/v, wheat gem oil in the range of 20-55% v/v, olive oil in the range of 15-45% v/v, castor oil in the range of 2-10% v/v, and cypress essential oil in the range of up to 5% v/v, with the balance of the composition at 100%.

In particular, the components of the preferred carrier oil afford additional therapeutic benefits to the patient treated with the preferred embodiment of the present invention. Sweet almond oil, olive oil, castor oil and wheat germ oil are readily absorbed by the skin, thereby transporting the cypress essential oil to sub-cutaneous layers of the dermis to effect its therapeutic effect on the affected area. Additionally, olive oil, sweet almond oil and wheat germ oil are emollients. Castor oil is alleged to have anti-microbial properties and wheat germ oil is rich in vitamins A, B1 and B2, D and E.

Other pharmaceutically or cosmetically acceptable vehicles are lotions, creams, ointments, gels, and solid formulations such as for example a wax-based stick. Other pharmaceutically or cosmetically acceptable vehicles can be formulated by those of ordinary skill in the art.

In one embodiment, the composition further comprises other pharmaceutically active agents in addition to cypress essential oil, provided that they do not detrimentally affect the desired effect on the treated area afforded composition. The term “pharmaceutically active agent” as used herein refers to a compound that has a cosmetic or therapeutic effect on the skin, for example, vitamins, antioxidants, essential oils, botanical or animal extracts, anti-microbial agents, and the like, and combinations thereof. Examples of vitamins include, but are not limited to, vitamin A, vitamin Bs such as vitamin B3, vitamin B5, and vitamin B12, vitamin C, vitamin K, and vitamin E and derivatives thereof.

Examples of essential oils include, but are not limited to, bergamot, clary sage, lavender, juniper, pine, marjoram, sandalwood, rosemary, frankincense, and citrus oils.

It is envisaged that a person skilled in the art will readily identify suitable pharmaceutically active agents which can be combined with the topical composition of the present invention.

In addition, the topically applied compositions of the present invention can contain conventional cosmetic additives, such as for example colourants and fragrances.

Further, the topically applied compositions of the present invention can contain preservatives, humectants, anti-microbial agents and the like to stabilize the topically applied composition and prolong its shelf life.

To prepare the topically applied compositions of the present invention, the cypress essential oil and, optionally, other pharmaceutically active agents are mixed with the pharmaceutically or cosmetically acceptable vehicle. The pharmaceutically or cosmetically acceptable vehicle acts as a diluent or carrier for other materials present in the composition, so as to facilitate their distribution and delivery to the affected area which requires treatment.

In one embodiment, the composition is topically applied to an area affected by striae distensae lesions at least once a week, such as once or twice a day. In one embodiment, the composition is topically applied to the affected area until the appearance of the striae distensae lesions has been improved, reduced, or minimized to the satisfaction of the user.

Typically, the composition is topically applied to the affected area twice daily for the first two weeks of treatment and then once daily, although a twice daily application or intermittent application every few days may provide satisfactory results in some cases. Clinically significant improvement in the appearance of the lesions is generally observed after three to six weeks of daily treatment, wherein the size and colour of the lesions are substantially reduced.

The following examples illustrate, but do not limit, the invention by detailing a preferred embodiment and the efficacy of the same in treating striae distensae lesions.

### EXAMPLE 1

The following composition was topically applied once daily to an extensive abdominal area of a post-natal woman exhibiting striae distensae lesions, resulting in a marked improvement (estimated at 95%) in the appearance of the lesions in three weeks.

Composition

| | |
|---|---|
| Sweet almond oil | 43.3% vol |
| Olive oil | 28.9% vol |
| Wheatgerm oil | 14.4% vol |
| Castor oil | 9.6% vol |
| Cypress essential oil | 3.8% vol |

### EXAMPLE 2

The first composition was topically applied once or twice daily to an extensive abdominal area of a post-natal woman exhibiting striae distensae lesions for a period of ten (10) days, and then the affected area was treated with once or twice daily topical applications of the second composition for a further period of 3-4 weeks, resulting in a marked improvement (estimated at 95%) in the appearance of the lesions after completion of the above treatment.

First Composition

| | |
|---|---|
| Sweet almond oil | 39.0% vol |
| Olive oil | 24.4% vol |
| Wheatgerm oil | 29.3% vol |
| Castor oil | 4.9% vol |
| Cypress essential oil | 1.2% vol |
| Juniper essential oil | 1.2% vol |

Second Composition

| | |
|---|---|
| Sweet almond oil | 40.0% vol |
| Olive oil | 25.0% vol |
| Wheatgerm oil | 30.0% vol |
| Castor oil | 2.5% vol |
| Cypress essential oil | 2.5% vol |

### EXAMPLE 3

The following composition was topically applied intermittently to an extensive area covering the abdomen and buttocks of a post-natal woman exhibiting striae distensae lesions, resulting in a marked improvement (estimated at 80%) in the appearance of the lesions.

Composition

| | |
|---|---|
| Sweet almond oil | 40.0% vol |
| Olive oil | 25.0% vol |
| Wheatgerm oil | 30.0% vol |
| Castor oil | 2.5% vol |
| Cypress essential oil | 2.5% vol |

### EXAMPLE 4

The following composition was topically applied once daily to an extensive lumbo-sacral area of an adolescent male exhibiting striae distensae lesions, resulting in an improvement (estimated at 60-70%) in the appearance of the lesions in three weeks.

Composition

| | |
|---|---|
| Sweet almond oil | 45% vol |
| Olive oil | 30% vol |
| Wheat germ oil | 15% vol |
| Castor oil | 7% vol |
| Cypress essential oil | 3% vol |

The following compositions may also be used in accordance with a preferred embodiment of the present invention.

### EXAMPLE 5

| | |
|---|---|
| Sweet almond oil | 60% vol |
| Olive oil | 20% vol |
| Wheatgerm oil | 15% vol |
| Castor oil | 2% vol |
| Cypress essential oil | 3% vol |

EXAMPLE 6

| | |
|---|---|
| Sweet almond oil | 55% vol |
| Olive oil | 25% vol |
| Wheatgerm oil | 15% vol |
| Castor oil | 2.5% vol |
| Cypress essential oil | 2.5% vol |

### EXAMPLE 7

| | |
|---|---|
| Sweet almond oil | 35% vol |
| Olive oil | 30% vol |
| Wheatgerm oil | 25% vol |
| Castor oil | 7% vol |
| Cypress essential oil | 3% vol |

### EXAMPLE 8

| | |
|---|---|
| Sweet almond oil | 30% vol |
| Olive oil | 40% vol |
| Wheatgerm oil | 20% vol |
| Castor oil | 7% vol |
| Cypress essential oil | 3% vol |

### EXAMPLE 9

| | |
|---|---|
| Sweet almond oil | 25% vol |
| Olive oil | 30% vol |
| Wheatgerm oil | 40% vol |
| Castor oil | 2.5% vol |
| Cypress essential oil | 2.5% vol |

### EXAMPLE 10

| | |
|---|---|
| Sweet almond oil | 20% vol |
| Olive oil | 55% vol |
| Wheatgerm oil | 20% vol |
| Castor oil | 2.5% vol |
| Cypress essential oil | 2.5% vol |

### EXAMPLE 11

| | |
|---|---|
| Sweet almond oil | 20% vol |
| Olive oil | 45% vol |
| Wheatgerm oil | 30% vol |
| Castor oil | 2.5% vol |
| Cypress essential oil | 2.5% vol |

### EXAMPLE 12

| | |
|---|---|
| Sweet almond oil | 20% vol |
| Olive oil | 30% vol |
| Wheatgerm oil | 45% vol |
| Castor oil | 2.5% vol |
| Cypress essential oil | 2.5% vol |

### EXAMPLE 13

Compositions falling within the range outlined below are envisaged to be topically applied to males or females who exhibit striae distensae lesions in accordance with the present invention.

Composition

| | | |
|---|---|---|
| Sweet almond oil | 36.2-41.3% | vol |
| Olive oil | 27.1-31.0% | vol |
| Wheatgerm oil | 24.8-31.6% | vol |
| Castor oil | 2.3-2.6% | vol |
| Cypress essential oil | 2.5-2.8% | vol |
| Rosehip oil | 0.2% | vol |
| Avocado oil | 0.2% | vol |

Numerous variations and modifications will suggest themselves to persons skilled in the relevant art, in addition to those already described, without departing form the basic inventive concepts. All such variations and modifications are to be considered within the scope of the present invention, the nature of which is to be determined from the foregoing description.

1. A topically applied composition for treatment of striae distensae lesions, the composition comprising a pharmaceutically or cosmetically acceptable vehicle for topical application and an effective amount of cypress essential oil.

2. The topically applied composition according to claim 2, wherein the cypress essential oil is present in an amount up to 5% by volume.

3. The topically applied composition according to claim 3, wherein the cypress essential oil is present in an amount between 1.0-4.0% by volume.

4. The topically applied composition according to any one of claims 1 to 3, wherein the pharmaceutically or cosmetically acceptable vehicle is a carrier oil.

5. The topically applied composition according to claim 4, wherein the carrier oil comprises sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, and vegetable oils such as for example safflower oil, sunflower oil, canola oil, soybean oil, and peanut oil, and combinations thereof.

6. The topically applied -composition according to claim 5, wherein the carrier oil is a mixture of sweet almond oil, wheat germ oil, olive oil, and castor oil.

7. The topically applied composition according to claim 5, wherein the topically applied composition comprises sweet almond oil in the range of 20-60% v/v, wheat germ oil in the range of 20-55% v/v, olive oil in the range of 15-45% v/v, castor oil in the range of 2-10% v/v, and cypress essential oil in the range of up to 5% v/v.

8. The topically applied composition according to any one of claims 1 to 3, wherein the pharmaceutically or cosmetically acceptable vehicles comprise lotions, creams, ointments, gels, and solid formulations such as for example a wax-based stick.

9. The topically applied composition according to claim 1, wherein the composition further comprises other pharmaceutically active agents in addition to cypress essential oil, provided that they do not detrimentally affect the desired effect on the treated area afforded by the cypress essential oil.

10. A method of treating striae distensae lesions to improve the appearance thereof comprising topically applying to an area affected by the lesions a composition according to claim 1.

11. The method of claim 10, wherein the composition is topically applied to an area affected by striae distensaestriae distensae lesions at least once a week.

12. The method of claim 11, wherein the composition is topically applied to the area affected by striae distensae lesions once or twice a day.

13. A method of improving the appearance of the striae distensae lesions comprising topically applying to an area affected by the lesions a composition according to claim 1.

14. A topically applied composition according to claim 1, including preparing a dermopharmaceutical or cosmetic to improve the appearance of the striae distensae lesions.
